(19) United States
(12) Patent Application Publication
(10) Pub. No.: US 2014/0214068 A1
(43) Pub. Date: Jul. 31, 2014

(54) CATHETER BALLOON AND FORMATION THEREOF

(71) Applicant: Edwards Lifesciences Corporation, Irvine, CA (US)

(72) Inventors: Yidong M. Zhu, Irvine, CA (US); Marlowe E. Patterson, Orange, CA (US); Dan C. Larsen, Sandy, UT (US)

(73) Assignee: Edwards Lifesciences Corporation, Irvine, CA (US)

(21) Appl. No.: 14/149,637

(22) Filed: Jan. 7, 2014

Related U.S. Application Data
(60) Provisional application No. 61/757,615, filed on Jan. 28, 2013.

(51) Int. Cl. A61M 29/02; A61M 25/10

(52) U.S. Cl. CPC A61M 29/02; A61M 25/1029
USPC 606/194; 264/534

(57) ABSTRACT

A process for forming a catheter balloon includes subjecting a tubular parison in a mold to molding fluid pressure. The resulting catheter balloon includes a balloon portion having two ends and tubular leg portions extending from either end. The ends of the balloon portion are tapered to the tubular leg portions. While in the mold, the tubular leg portions are drawn axially sufficiently to form permanent creases in the tapered ends. This drawing may be sufficient to cause the material of the tapered ends to exceed the yield strength, particularly with the balloon material in a malleable state. Anomalies on tapered parts of the mold may include ribs or spiral ribs to assist in the creasing process.

### CROSS-REFERENCE TO RELATED APPLICATIONS

This application claims the benefit of U.S. Patent Application No. 61/757,615, filed Jan. 28, 2013, the entire disclosure of which is incorporated herein.

### BACKGROUND

The field of the present application is balloons for medical dilation procedures.

Balloons associated with catheters for percutaneous procedures are now quite common; Such procedures include angioplasty, valvuloplasty and urological dilation which employ a balloon with or without companion equipment such as expandable stents to expand in the body. Percutaneous procedures can eliminate the need, risk and expense for more conventional surgical procedures and greatly reduce recovery time.

Balloons are employed with catheters which are thin, flexible lengths of tubing that are fed percutaneously through an arterial system to a location requiring wall or port expansion or lining material compression. One or more balloons is appropriately placed along the length of the tubing, typically near the tip. The balloons are introduced in a contracted state for placement in a body passageway such as the lumen of a blood vessel, a urological passageway, or the like. Fluoroscopic guidance typically assists in the appropriate threading and placement of the catheter and of the balloon mounted thereon. A guide wire typically extends from the distal end of the catheter and is able to move axially of the catheter to assist in the proper placement thereof. Further, sheaths are frequently concentrically arranged on the catheters and extend over the undeployed balloons. When the sheath is drawn axially from over the balloon, the balloon can then be expanded to a taut or optionally a distended state depending on the elasticity and other properties of the balloon material.

Once the balloon has been expanded to perform the appropriate procedure, the balloon is collapsed. Such a collapse can be provided by release of fluid pressure within the balloon, possible vacuum drawn on the balloon and/or reextension of the sheath over the balloon. The extension of a sheath over the balloon is often difficult to accomplish, requiring an inconvenient level of force. Difficulties using a sheath are further compounded by the large scale of such devices used in percutaneous widening of a stenotic heart valve.

The balloons employed in such medical procedures are generally polymeric materials. Polyethylene terephthalates, polyvinyl chlorides and cross-linked polyethylenes as well as other materials are known to be employed in the fabrication of balloons for percutaneous medical procedures.

These materials, whether non-distensible or distensible, are considered highly reliable, particularly in comparison with open chest cavity procedures, for example. Even so, failure concerns must be addressed. Catheter balloons have the possibility of tearing under load or manipulation. Such failure can occur either along a longitudinal or a circumferential tear. Longitudinal tears are considered relatively safe. Circumferential tears, on the other hand, are considered clinically unsafe. Retraction into a sheath can result in circumferential tears under adverse circumstances. However, increasing the wall thickness of such balloon materials increases the invasive aspect of the device. Thus, conflicting design criteria, particularly as the diameter of the expanded balloon increases, must be reconciled.

Disclosures directed to existing balloon technology and balloon fabrication are found in U.S. Pat. Nos. 7,128,868; 6,500,148; 6,428,568; 5,350,361; and 5,147,302, the disclosures of which are incorporated herein by reference.

### SUMMARY

The present application is directed to balloons for employment in percutaneous medical procedures and to processes for the fabrication of such balloons. Designs are employed to reduce the forces necessary for the resheathing of a used balloon prior to extraction and to avoid circumferential failures.

A first aspect provides a process for forming a catheter balloon includes molding such a balloon from a tubular parison. The resulting balloon includes a balloon portion having two ends and tubular leg portions at either end of the balloon portion. At least one of the ends of the balloon portion is tapered to the respective tubular leg portion. The molded balloon is then axially drawn partially from the mold at least at one end in a manner sufficient to form permanent creases in that tapered end.

A second aspect of provides a process for forming a catheter balloon includes molding such a balloon from a tubular parison. The resulting balloon includes a balloon portion having two ends and tubular leg portions at either end of the balloon portion. At least one of the ends of the balloon portion is tapered to the respective tubular leg portion. Longitudinal anomalies in a tapered portion of the mold, which may be longitudinal ribs or longitudinally extending spiral ribs, further influence the molding process as the balloon is axially drawn,

A third aspect provides a process for forming a catheter balloon includes molding such a balloon from a tubular parison. The resulting balloon includes a balloon portion having two ends and tubular leg portions at either end of the balloon portion. At least one of the ends of the balloon portion is tapered to the respective tubular leg portion. The axial drawing of the molded balloon is sufficient to exceed the yield strength, the process being undertaken with the balloon in a malleable state.

A fourth aspect provides a catheter balloon includes a tubular balloon portion, tapered ends at either end of the tubular balloon portion and tubular legs extending from the tapered ends. The tapered ends include creases extending longitudinally of at least a portion of the tapered ends and being of a smaller mass per unit axial length than the tubular legs.

A fifth aspect provides any of the foregoing aspects employed in combination to greater advantage.

Thus, it is a principal object to provide improved catheter balloons capable of facile sheathing and methods of fabrication. Other and further objects and advantages will appear hereinafter.

### BRIEF DESCRIPTION OF THE DRAWINGS

FIG. 1 illustrates a side view of a catheter balloon associated with a catheter with portions broken away for clarity.

FIG. 2 illustrates a cross-sectional side view of a catheter balloon in a forming mold.

FIG. 3 illustrates a second embodiment of a cross-sectional side view of a portion of a forming mold for a catheter balloon.

FIG. 4 illustrates a third embodiment of a cross-sectional side view of a portion of a forming mold for a catheter balloon.

### DETAILED DESCRIPTION OF CERTAIN EMBODIMENTS

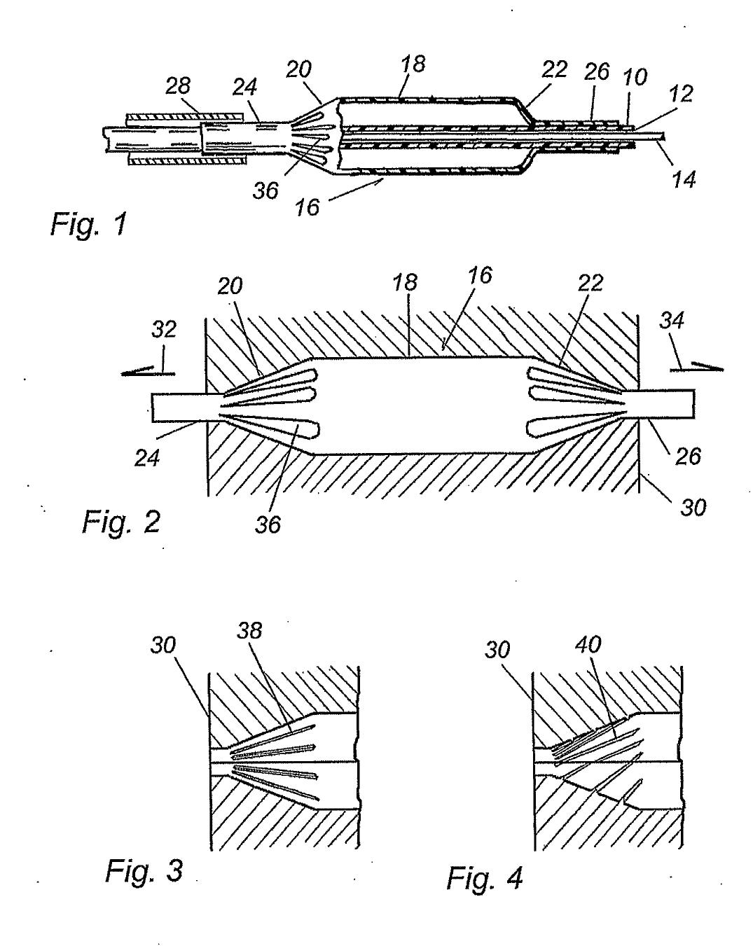

Turning in detail to the drawings, an inflated balloon associated with a catheter is illustrated in FIG. 1. The catheter 10, illustrated only in part, has a tube with a lumen 12 therethrough. A guide wire 14 extends through the lumen 12 to assist in placement of the catheter 10. The catheter 10 also has at least one additional lumen, not shown, to feed fluid pressure for balloon inflation. A balloon 16 is shown to be positioned on the catheter 10 and is sealed at both ends.

The balloon 16 includes a cylindrical balloon portion 18 with two ends 20, 22 which are tapered to tubular leg portions 24, 26. The entire balloon 16 has a passage therethrough with the balloon 16 having been molded from a tubular parison. In FIG. 1, the balloon is shown in an inflated state. Typically a balloon 16 is deployed percutaneously in a deflated state and frequently with an axially movable sheath 28 over the collapsed balloon 16. The sheath 28 is withdrawn from about the balloon 16 before inflation. The sheath 28 may be replaced over the balloon 16 before retraction of the catheter 10 from the vascular system. Balloon deflation precedes resheathing.

In fabricating the balloon 16 from at tubular parison, the balloon 16 is placed in a mold 30 such as roughly illustrated in FIG. 2 with a balloon 16 in place. The mold 30 has the appropriate shape of the resulting balloon portion 18 with the tapered ends 20, 22 and passageways for the tubular leg portions 24, 26 and is split through the centerline of the resulting balloon 16. The tubular leg portions 24, 26 are actually unexpanded portions of the parison. Pressurized fluid is injected into the parison while in the mold cavity until the parison expands within the mold 30 to create the balloon 16. With release of the fluid, the mold 30 may be separated and the balloon 16 extracted.

With the pressure released and before the balloon 16 is removed from the mold 30, one or both ends of the tubular leg portions 24, 26 are axially pulled as indicated by the arrows 32, 34. Pulling the leg portions 24, 26 axially draws the balloon 16 partially from the mold 30. As the balloon 16 is moving into the tubular sections of the mold 30 reserved for the leg portions 24, 26, the tapered ends 20, 22 are radially compressed. At the same time, there is understood to be a drawing action beyond the yield point of these ends, 20, 22. This action may be undertaken with the balloon 16 still in a malleable state at a temperature determined by the type of material employed. The effect of this action is to create permanent creases 36 extending longitudinally through part of or all of the tapered ends 20, 22. Once permanent creases 36 have been defined, the tubular leg portions 24, 26 are released and the mold 30 is opened.

The intent of the creases 36 is to facilitate resheathing of the balloon 16 with a reduced force. The creases 36 provide a predisposition for the balloon 16 to appropriately fold and be drawn into the sheath 28. Through testing, it has been demonstrated that the amount of force required to resheath the balloon 16 drops by in excess of one-half when such creases 36 are employed over uncreased balloons. This is understood to occur because the creases 36 are already formed in the material and less force is required to refold the permanently creased balloon. Depending upon the malleability of the material at the temperature in the mold, the creases 36 may extend more or less into the tapered ends 20, 22 and possibly even onto the cylindrical balloon portion 18. It is believed that the drawing of the balloon 16 from the tubular ends of the mold 30 results in an elongation of the tapered end section or sections 20, 22. As such, the mass per unit length decreases in this region or regions as compared with the portions of the balloon 16 where longitudinal extension does not occur.

Where regular patterns are desired, such as for the creation of regular pleats in the balloon 16, variations in the mold 30 at the tapered sections may be undertaken. FIG. 2 illustrates a system where the mold 30 has smooth truncated conical end sections to define the tapered ends 20, 22. In FIG. 3, longitudinal ribs 38 are formed about this conical section of the mold 30. When appropriately spaced, it is anticipated that regular pleats can be generated as the balloon 16 is drawn partially from the mold 30 over the anomalies. Naturally, during the original formation of the balloon 16 from the parison, beginning creases will be formed by the ribs 38 even before the balloon 16 is drawn. In other embodiments, the ribs 38 are arranged in a different pattern, for example, with uneven spacing therebetween.

Another embodiment of the mold 30 is illustrated in FIG. 4 where spiral ribs 40 extend both longitudinally and rotationally about the tapered sections of the mold 30 corresponding to the formed tapered ends 20, 22 of the balloon portion 18. In this circumstance, drawing of the balloon 16 partially from the mold 30 might be accomplished with consistent rotation in keeping with the spiral ribs 40.

With any of the foregoing embodiments, folding of the balloon 16 to assemble the catheter 10 with the sheath 28 before use is undertaken. Attention is paid to the creases 36 that have been defined in the tapered end portions 20, 22 of the balloon portion 18 in that process.

Thus, an improved catheter balloon having permanent creases to reduce the force required in resheathing a balloon after expansion has been disclosed. These creases are further understood to inhibit circumferential splitting of the balloon 16. While embodiments and applications have been shown and described, it would be apparent to those skilled in the art that many more modifications are possible without departing from the concepts herein. The disclosure, therefore is not to be restricted except in the spirit of the appended claims.

What is claimed is:

1. A process for forming a catheter balloon comprising the steps of:
molding a catheter balloon in a mold from a tubular parison, the molded catheter balloon including a balloon portion having two ends and tubular leg portions at either end of the balloon portion, at least one of the ends of the balloon portion being tapered to the respective tubular leg portion;
axially drawing the molded catheter balloon partially from the mold at least at one end sufficiently to form permanent creases in the at least one tapered end.

2. The process of claim 1, the step of molding including using molding fluid pressure.

3. The process of claim 2 further comprising the step of:
at least partially releasing the molding fluid pressure within the molded catheter balloon after molding and before axially drawing the molded catheter balloon.

4. The process of claim 1, the step of axially drawing including axially drawing the molded catheter balloon partially from the mold at both of the ends of the balloon portion, both of the ends of the balloon portion being tapered.

5. A process for forming a catheter balloon comprising the steps of:
molding a catheter balloon in a mold from a tubular parison using molding fluid pressure, the molded catheter balloon including a balloon portion having two ends and tubular leg portions at either end of the balloon portion, at least one of the ends of the balloon portion being tapered to the respective tubular leg portion;
axially drawing the molded catheter balloon partially from the mold at least at one end sufficiently to form permanent creases in the at least one tapered end with the at least one tapered end exceeding the yield strength and being at a malleable temperature.

6. The process of claim 5 further comprising the step of:
at least partially releasing the molding fluid pressure within the molded catheter balloon after molding and before axially drawing the molded catheter balloon.

7. The process of claim 5, the step of axially drawing including axially drawing the molded catheter balloon partially from the mold at both of the ends of the balloon, both of the ends of the balloon portion being tapered.

8. A process for forming a catheter balloon comprising the steps of:
molding a catheter balloon in a mold from a tubular parison using molding fluid pressure, the molded catheter balloon including a balloon portion having two ends and tubular leg portions at either end of the balloon portion, at least one of the ends of the balloon portion being tapered to the respective tubular leg portion;
axially drawing the molded catheter balloon partially from the mold at least at one end over longitudinal anomalies in a tapered portion of the mold in which the at least one tapered end of the balloon portion is formed sufficiently to form permanent creases in the at least one tapered end.

9. The process of claim 8 further comprising the step of:
at least partially releasing the molding fluid pressure within the molded catheter balloon after molding and before axially drawing the molded catheter balloon.

10. The process of claim 8, the step of axially drawing including the longitudinal anomalies being longitudinal ribs.

11. The process of claim 8, the step of axially drawing including rotating the tubular leg portions with the longitudinal anomalies being spiral ribs, the rotation being consistent with the spiral ribs.

12. The process of claim 8, the step of axially drawing including axially drawing the molded catheter balloon partially from the mold at both of the ends of the balloon portion, both of the ends of the balloon portion being tapered.

13. The process of claim 8, the step of axially drawing including the at least one tapered end exceeding the yield strength and being at a malleable temperature.

14. A catheter balloon comprising:
a tubular balloon portion;
tapered ends at either end of the tubular balloon portion including creases extending longitudinally of at least a portion of the tapered ends;
a tubular leg extending from each of the tapered ends, the tapered ends having a smaller mass per unit axial length than the tubular legs.

15. The catheter balloon of claim 14, the creases being regular pleats.
